(19) United States
(12) Patent Application Publication
(10) Pub. No.: US 2011/0245639 A1
(43) Pub. Date: Oct. 6, 2011

(54) PHYSIOLOGICAL PARAMETER STATISTICAL PROCESSING AND DISPLAY

(76) Inventors: Oleg Gonopolskiy, West Bloomfield, MI (US); Ronald A. Widman, Macomb, MI (US); Bryan Burke, Birmingham, MI (US); Michael D. Wider, Pleasant Ridge, MI (US); Arik Anderson, Birmingham, MI (US)

(21) Appl. No.: 13/076,011

(22) Filed: Mar. 30, 2011

Related U.S. Application Data
(60) Provisional application No. 61/319,104, filed on Mar. 30, 2010.

(51) Int. Cl. A61B 5/145

(52) U.S. Cl. 600/323

(57) ABSTRACT

A patient monitoring system has a processor and a spectrophotometric sensor. The sensor is configured to be affixed to a patient to communicate signals associated with real time spectrophotometric measurements to the processor. The system further includes memory for storing data and computer instructions. The processor is configured to execute instructions stored in the memory to calculate a trend statistic based on a group of the signals received from the sensor. The processor is further configured to execute instructions stored in the memory to cause real time information associated with the spectrophotometric measurements and information associated with the trend statistic to be displayed on a visual user interface.

### CROSS-REFERENCE TO RELATED APPLICATIONS

This Application claims the benefit of U.S. Provisional application No. 61/319,104 filed on Mar. 30, 2010, the entirety of which is hereby incorporated by reference.

### BACKGROUND

Patient monitoring systems for sensing, monitoring and displaying the blood oxygen saturation level (rSO₂) of a region of a patient are known, including the commercially-available INVOS® system from Somanetics Corporation in Troy, Mich., now owned by Covidien, headquartered in Mansfield, Mass. Such systems commonly include a sensor configured to be temporarily affixed to a patient and in communication with a processor, which is configured to receive signals from the sensor and calculate an rSO₂ value. The systems commonly calculate an rSO₂ of a monitored region of the patient on a periodic basis, for example, every five seconds. Conventionally, the most current “real time” rSO₂ value and a historical graph of some number of prior real time rSO₂ values may be displayed on a computer monitor so that a caregiver could read and assess the rSO₂ data.

It has been observed by the inventors, though, that displaying the real time rSO₂ value and historical rSO₂ values alone may not be optimal for a caregiver to properly assess the impact of blood oxygen saturation on the patient, particularly (though not exclusively) in neonates. Some patients, especially neonates, exhibit large variations in tissue oxygen (O₂) delivery, which can be dependent on gestational age, day of life, and on the tissue or organ monitored. As a result, a progressive change in the rSO₂ value, which may be indicative of an impending biological catastrophe, may be obscured by the high variability of the real-time rSO₂ values.

Accordingly, the inventors hereof have identified a need for an improved patient monitoring and display system for rSO₂ levels that calculates and displays additional data to improve the information available to a caregiver assessing the blood oxygen saturation condition of a patient.

### SUMMARY

An improved patient monitoring system for monitoring rSO₂ of a patient is disclosed. The improved system displays historical and current real time rSO₂ values for the patient. Additionally, the system calculates and displays statistical trending data, such as a trailing statistical average, of the real time rSO₂. The combined display of real time rSO₂ values and statistical trend data of the rSO₂ values better enables a caregiver to assess a patient's blood oxygen saturation condition, including predicting impending biological catastrophes, particularly in patients (such as neonates) who have high variability in O₂ delivery.

### BRIEF DESCRIPTION OF THE FIGURES

FIG. 1 is an illustrative example of a system according to an embodiment, as used in one exemplary environment to perform spectrophotometric cerebral oximetry.

FIG. 2 illustrates an exemplary screenshot of the display of the patient monitoring system configured to display real time blood oxygen saturation information and a statistical trend value, both on a plotted line graph over time.

### DETAILED DESCRIPTION

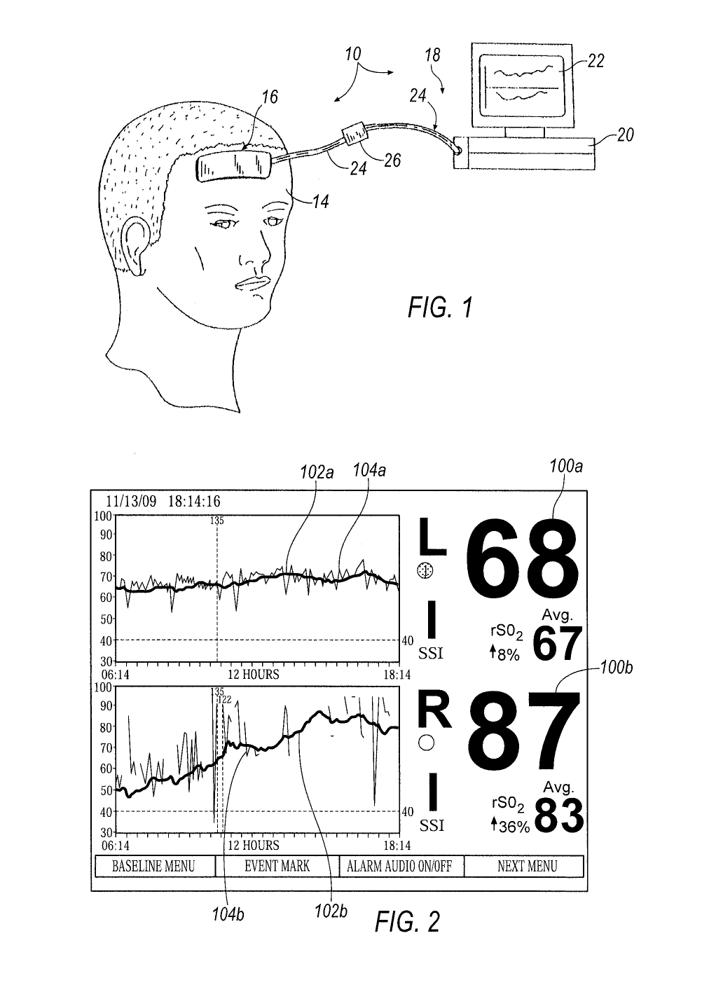

FIG. 1 illustrates an exemplary environment for implementation of a system 10 for monitoring rSO₂ of a patient. The system 10 has a spectrophotometric apparatus 18 connected to a sensor 16 through an electrical cable 24. The electrical cable 24 may include a signal amplifier 26. The spectrophotometric apparatus 18 is a computer or other processor-based computing device 20 and a monitor or other visual display device 22. The computing device 20 includes customary memory devices that store data and algorithm instructions and a processor that executes algorithm instructions. The sensor 16 takes spectrophotometric readings of the monitored region and generates corresponding representative electrical signals, which are conveyed to the computing device 20. The computing device 20 processes the signals and causes data to be displayed on the monitor 22.

Periodically, the computing device 20 calculates an rSO₂ value from the electrical signals. The calculated real time rSO₂ value is numerically displayed on the monitor 22. Additionally, a certain number of historical real time rSO₂ values are graphically plotted to generate a line graph of the historical real time rSO₂ values over time. From these two displays, a caregiver can observe the current rSO₂ level of the patient, as well as the historical rSO₂ levels.

Further, computing device 20 calculates a trend statistic of the real time rSO₂ values. One such trend statistic is a trailing average of the real time rSO₂ values. A person of ordinary skill in the art understands how to calculate a trailing average value from a group of rSO₂ values. In general, an average of all of the non-zero rSO₂ values calculated for a particular period of time, e.g., the last 60 minutes, is calculated each time a new real time rSO₂ value is calculated. Each calculated average value is plotted to generate a line graph of average rSO₂ values over time, which is displayed on the monitor 22 concurrently with the numerical representation of the current real time rSO₂ value and the line graph of the historical real time rSO₂ values. Other known trend statistics may be used instead of a trailing average value, such as a trailing median value, and they may be displayed in various ways other than a line graph. The object is to calculate and display a trend statistic that provides a caregiver with information from which the trend of the real time rSO₂ values can be assessed.

FIG. 2 illustrates an exemplary display on a monitor 22 showing the real time rSO₂ values and the statistical trend data for two different channels. References 100a and 100b are directed to the numerical representation of the current real time rSO₂ value for the first and second channels, respectively; lines 104a and 104b are the graphical representations of the historical real time rSO₂ values, plotted over time, for the first and second channels, respectively; line 102a and 102b are the graphical representations of statistical trend data, e.g., the trailing average values, plotted over time for the first and second channels, respectively. Other configurations and arrangements of the illustrated regions on the monitor 22 are contemplated and within the scope of invention.

The exemplary embodiment described herein has several advantages over known blood oxygen saturation monitoring systems. For example, a trend statistic, such as a trailing average or median, can alert a caregiver to slowly progressive changes that presage impending events, including catastrophic biologic changes. This is counterintuitive since it would seem more appropriate to watch the real time measurements than to look at a trend statistic. However, where the perfusion distribution of the patient, such as a neonate, is highly variable, progressive average change of the data can be obscured by the erratic nature of the real time values. Displaying a trend statistic assists the caregiver in identifying such progressive average changes. On the other hand, it remains useful to display the real time values as well. The real time values allow the caregiver to determine if the trend statistic represents mostly signal dropout coupled with consistently low readings or if it is just the normal wide variation giving the same low average blood oxygen saturation values.

The combined use of real time rSO₂ values and trend statistics is beneficial as illustrated by the following examples. An rSO₂ profile of mostly rSO₂ of 15-20 mmHg with intermittent periods of 35-45 mmHg can evidence a different clinical condition from prolonged periods of mostly 20-25 mmHg with no periods higher. But both could present the same trend statistic (e.g., a trailing average) while the real time data would highlight the difference. Conversely, presenting the data as a rolling average in combination with the real time data is critical so that if there is a sudden catastrophic change it will not be obliterated by the average graph. This is exemplified in a situation where PaCO₂ suddenly drops due to over ventilation causing a dramatic drop in the cerebral blood flow¹. ¹PaCO₂ is the partial pressure of carbon dioxide in arterial blood, measured by analyzing an arterial blood sample on a blood gas machine. Normal range is 35-45 mmHg and increases in PaCO₂ selectively raise cerebral blood flow by about 2-3% per mmHg and vice versa.

An exemplary application of the above-described embodiment is directed to detecting necrotizing entercolitis (“NEC”) in neonates. NEC in neonates may be predicted by caregivers based on the degree of variability of rSO₂ in the gut of a neonate.

In addition to display of averaged values, an exemplary approach is described where a measure of variance is ascribed to the averaged data epoch. This measure of variance could be the actual statistical variance, the standard deviation, the confidence interval, standard error or some other measure of variability of the data, hereinafter “index of variability.” Variability over short (0-60 seconds) and medium (1-30 minutes) time frames is inherent to physiological systems and can indicate the robustness of those systems. The index of variability can be used to track both short- and medium-term variability depending on the length of the averaging epochs and the method used to calculate the index. Because different areas of the body exhibit differing blood flow rates, the time frames, epoch lengths, and methods used to calculate the variability index can be adjusted based on expected flow in various organs or body areas. This adjustment can be user selectable or can be automatically invoked based on the label assigned to a specific channel indicating its sensor location or typical flow rates.

Variability in certain physiological systems can change based on factors other than the patient's well-being. For example, variations of the hemodynamics of the splanchnic circulation can change significantly during pre- and post-prandial conditions. Likewise, variations in cerebral blood flow can increase significantly if cerebral perfusion pressure falls to a level close to or below the lower limit of autoregulation. Premature infants exhibit very high levels of variability in some organ beds such as the splanchnic bed during the first weeks of life. Therefore, the patient monitor disclosed herein can change the method of calculation, the length of data epochs, or the thresholds used for alerting caregivers based on demographics, gestational age, location of the measurement, feeding status or other measures or parameters to allow the system to adjust to varying conditions and demographics.

The index of variability can be displayed in several unique ways. For example, in one exemplary implementation, dotted lines above and below the trend line of the average value can indicate variance above and below the mean value. The areas above and below the mean may be filled in with a transparent color such that objects below are still visible. Further, a series of whiskers or error bars may be added to the averaged trend to indicate the magnitude of variability above and below the mean.

Changes in the index of variability can be tracked over time to indicate basic changes in the well-being of the patient. As variability decreases, in most cases the overall well-being of the patient is declining. Likewise, as variability increases, well-being is usually improving. Therefore, changes in variability beyond a fixed or user-adjustable threshold can be used to alert caregivers to changes that may reflect changes in patient condition. Additionally, real time values that remain significantly outside the limits of variability for a preset or adjustable time period may also trigger an alert or message to indicate a major change in the patient's condition. Indication of significant changes in the index of variability can be indicated on the trend through color changes, drawing the user's attention to the change as it occurs. Alternately, changes in variability can trigger a message on the screen or can be used to activate an audible alert to warn the user that a change is occurring.

While the index of variability can extract information on significant changes to the magnitude of variations, another implementation can process data in a way that extracts information on the frequency of variations. By observing data in the frequency domain, significant changes in the power and frequency of variability can be observed in real time. The patient monitor described herein is configured to convert epochs of data to the frequency domain using a method such as Fourier transformation where the power of variability is plotted against the frequency of that variation. Using this technique, significant changes in either power or dominant frequency of variations can be tracked and changes greater than a threshold can be used to trigger an alert as described previously.

With regard to the processes, systems, methods, heuristics, etc. described herein, it should be understood that, although the steps of such processes, etc. have been described as occurring according to a certain ordered sequence, such processes could be practiced with the described steps performed in an order other than the order described herein. It further should be understood that certain steps could be performed simultaneously, that other steps could be added, or that certain steps described herein could be omitted. In other words, the descriptions of processes herein are provided for the purpose of illustrating certain embodiments, and should in no way be construed so as to limit the claimed invention.

Accordingly, it is to be understood that the above description is intended to be illustrative and not restrictive. Many embodiments and applications other than the examples provided would be apparent upon reading the above description. The scope of the invention should be determined, not with reference to the above description, but should instead be determined with reference to the appended claims, along with the full scope of equivalents to which such claims are entitled. It is anticipated and intended that future developments will occur in the technologies discussed herein, and that the disclosed systems and methods will be incorporated into such future embodiments. In sum, it should be understood that the invention is capable of modification and variation.

All terms used in the claims are intended to be given their broadest reasonable constructions and their ordinary meanings as understood by those knowledgeable in the technologies described herein unless an explicit indication to the contrary in made herein. In particular, use of the singular articles such as “a,” “the,” “said,” etc. should be read to recite one or more of the indicated elements unless a claim recites an explicit limitation to the contrary.

1. A patient monitoring system, comprising:
a processor;
a spectrophotometric sensor configured to be affixed to a patient and configured to communicate signals associated with real time spectrophotometric measurements to said processor;
memory for storing data and computer instructions;
said processor configured to execute said instructions to calculate a trend statistic based on a group of said signals; and
said processor configured to execute said instructions to cause real time information associated with said spectrophotometric measurements and information associated with said trend statistic to be displayed on a visual user interface.

2. The system of claim 1, wherein said trend statistic is selected from the group of: a trailing average and a trailing median.

3. The system of claim 1, wherein said processor is configured to execute instructions that enable a user of the system to change a method of calculating the trend statistic.

4. The system of claim 1, wherein the processor is configured to execute instructions to enable a user to change a length of data epochs.

5. The system of claim 1, wherein said processor is further configured to execute instructions that enable a user to set a threshold value that triggers an indicator indicative of a condition of the patient based on said spectrophotometric measurements.

6. The system of claim 5, wherein said thresholds are based on at least one of: demographics, gestational age, location of the measurement and feeding status of the patient.

7. The system of claim 1, wherein said processor is further configured to execute instructions to calculate variance statistics associated with said trend statistic and to display said variance statistics on said visual user interface.

8. The system of claim 1, wherein said visual user interface is a monitor.

9. The system of claim 1, wherein said real time information associated with said spectrophotometric measurements are graphically displayed.

10. A patient monitoring system, comprising:
a processor;
a spectrophotometric sensor configured to be affixed to a patient and configured to communicate signals associated with real time spectrophotometric measurements to said processor;
memory for storing data and computer instructions;
said processor configured to execute said instructions to calculate a trailing average value of said measurements; and
said processor configured to execute said instructions to cause real time information associated with said spectrophotometric measurements and information associated with said trend statistic be plotted in the form of separate line graphs concurrently on monitor.
